(19) United States
(12) Patent Application Publication
(10) Pub. No.: US 2020/0352989 A1
(43) Pub. Date: Nov. 12, 2020

(54) METHOD FOR PROMOTING LIPID METABOLISM OR ASSISTING IN BODY WEIGHT CONTROL

(71) Applicant: Maxluck Biotechnology Corp., Taipei City (TW)

(72) Inventor: Tsung-Sheng CHEN, Taipei City (TW)

(21) Appl. No.: 16/858,774

(22) Filed: Apr. 27, 2020

(30) Foreign Application Priority Data
May 8, 2019 (TW) 108115798

(51) Int. Cl. A61K 33/24; A61K 47/64; A61K 47/18; A61K 31/145; A61K 31/355; A61K 31/4415

(52) U.S. Cl. CPC A61K 33/24; A61K 47/644; A61K 31/4415; A61K 31/145; A61K 31/355; A61K 47/183

(57) ABSTRACT

A method for promoting lipid metabolism or assisting in body weight control is disclosed, which includes: administering a composition to a subject in need thereof. The composition includes: a trivalent chromium complex being a complex of a trivalent chromium compound and a lactoferrin; and taurine or a derivative thereof.

### CROSS REFERENCE TO RELATED APPLICATION

This application claims the benefits of the Taiwan Patent Application Serial Number 108115798, filed on May 8, 2019, the subject matter of which is incorporated herein by reference.

### BACKGROUND OF THE INVENTION

### 1. Field of the Invention

The present disclosure relates to a method for promoting lipid metabolism or assisting body weight control and, more particularly, to a method for preventing or reducing body fat production in order to assist body weight control.

### 2. Description of Related Art

Nowadays, obesity is a common problem that every country endeavors to address. A person who is overweight or obese has a high risk of suffering from various diseases, such as hypertension, diabetes, cardiovascular disease, stroke, arthritis and dyslipidemia, and obesity even increases the mortality. In the medical profession, the use of drug or surgery for actively controlling obesity is recognized, while both of them result in some complications or sequelae. Although there are many weight loss drugs have undergone clinical trials, they are banned from the market several years later due to side effects. For example, the ingredient “Sibutramine” in Reductil increases the risk of cardiovascular disease.

The main reason for gaining weight is that body cannot use the excess calories after basic metabolism, and thus the excess calories are converted into lipids in the fat tissues. For the purpose of losing weight by using the lipids in the fat tissues, it is required to reduce calorie intake and increase the energy metabolism in the body. However, it takes time to undergo these processes and cannot be done at once. The body performs a feedback mechanism when it feels the energy loss in a large scale, in which the body will try to remain the storage of lipids no matter how. Therefore, it will increase the period of time for weight control, and most people will give up due to the long process.

In view of this, there is an urgent need to develop a composition that can effectively control the formation of body fat to achieve the purpose of weight control.

### SUMMARY OF THE INVENTION

The present disclosure provides a composition that promotes lipid metabolism to avoid or reduce the formation of body fat or reduce fat accumulation. The present disclosure further provides a composition assisting in weight control to achieve the effect of reducing body weight, maintaining body weight or slowing down the increase in body weight.

The present disclosure provides a composition for promoting lipid metabolism or assisting in weight control, comprising: a trivalent chromium complex being a complex of a trivalent chromium compound and a lactoferrin; and a taurine or a derivative thereof.

When the composition of the present disclosure is applied to a subject (for example, human or animal) in need, the chromium element in the composition can be effectively accumulated, by the synergistic effect of the trivalent chromium complex and the taurine or the derivative thereof, in insulin-sensitive tissues or organs (for example, liver, muscle or fat) to enhance the activity of insulin, thereby facilitating the energy metabolism. Particularly, even the subject does not produce extra physical activity or the subject maintains the same caloric intake, the chromium element in the composition can be effectively accumulated in insulin-sensitive tissues or organs to enhance lipid metabolism or inhibit lipid production, thereby controlling weight.

The trivalent chromium complex is an organic chromium complex. The transfer of the trivalent chromium complex between insulin-sensitive tissues or organs has impact on the energy transmission and metabolism of cells, facilitating the change in the allocation of the body fat and protein. In addition, the accumulation of taurine in cells depends on the taurine transporter, and the taurine transporter accelerates the speed of composition of the present disclosure (especially the trivalent chromium complex of the composition) for entering the cells, such that the chromium element in the trivalent chromium complex can be effectively accumulated in the insulin-sensitive tissues and organs.

In the composition of the present disclosure, a content of chromium in the trivalent chromium complex may be in a range from 0.1 to 1 parts by weight, and a content of taurine or the derivative thereof is in a range from 1,000 to 10,000 parts by weight. The content of chromium in the trivalent chromium complex is preferably in a range from 0.1 to 0.5 parts by weight, more preferably 0.2 to 0.4 parts by weight. The content of taurine or the derivative thereof is preferably in a range from 2,000 to 8,000 parts by weight, more preferably 3,000 to 6,000 pats by weight. In one embodiment of the present disclosure, the content of chromium is 0.4 parts by weight, and the content of taurine is 3,000 parts by weight. However, the present disclosure is not limited thereto.

In the composition of the present disclosure, a content of chromium of a trivalent chromium compound may be in a range from 0.1 to 1 parts by weight, a content of lactoferrin may be in a range from 1 to 20 parts by weight, and a content of taurine or the derivative thereof is in a range from 1,000 to 10,000 parts by weight. The content of taurine or the derivative thereof is preferably in a range from 2,000 to 8,000 parts by weight, more preferably 3,000 to 6,000 pats by weight. In one embodiment of the present disclosure, the content of chromium of the trivalent chromium compound is 0.4 parts by weight, the content of lactoferrin is 5 parts by weight, and the content of taurine is 3,000 parts by weight. However, the present disclosure is not limited thereto.

Optionally, the composition of the present disclosure may comprise glutamine. A content of glutamine may be in a range from 1,000 to 50,000 parts by weight. The content of glutamine preferably is in a range from 1,000 to 30,000 parts by weight, more preferably 5,000 to 15,000 parts by weight. In one embodiment of the present disclosure, the content of glutamine is 10,000 parts by weight. However, the present disclosure is not limited thereto.

Optionally, the composition of the present disclosure may further comprise cysteine. A content of cysteine may be in a range from 100 to 5,000 parts by weight, preferably 300 to 2,000 parts by weight, more preferably 500 to 1,000 parts by weight. In one embodiment of the present disclosure, the content of cysteine is 1,000 parts by weight. However, the present disclosure is not limited thereto.

Optionally, the composition of the present disclosure further may comprise glycine. A content of glycine may be in a range from 300 to 5,000 parts by weight, preferably 500 to 3,000 parts by weight, more preferably 1,000 to 2,000 parts by weight. In one embodiment of the present disclosure, the content of glycine is 1,000 parts by weight. However, the present disclosure is not limited thereto.

Optionally, the composition of the present disclosure may further comprise vitamin E. A content of vitamin E may be in a range from 1 to 30 parts by weight, preferably 3 to 20 parts by weight, and more preferably 6 to 12 parts by weight. In one embodiment of the present disclosure, the content of vitamin E is 10 parts by weight. However, the present disclosure is not limited thereto.

Optionally, the composition of the present disclosure may further comprise vitamin B6. A content of vitamin B6 may be in a range from 0.1 to 10 parts by weight, preferably 0.1 to 5 parts by weight, and more preferably 0.5 to 1 parts by weight. In one embodiment of the present disclosure, the content of vitamin B6 is 1 parts by weight. However, the present disclosure is not limited thereto.

Optionally, the composition of the present disclosure may further comprise whey protein. A content of whey protein may be in a range from 1,000 to 30,000 parts by weight, preferably 3,000 to 20,000 parts by weight, and more preferably 5,000 to 10,000 parts by weight. In one embodiment of the present disclosure, the content of whey protein is 10,000 parts by weight. However, the present disclosure is not limited thereto.

In a composition of the present disclosure, the trivalent chromium compound may be chromium (III) chloride hexahydrate, chromium (III) chloride, chromium (III) acetate, chromium (III) nitrate, chromium (III) oxide, chromium (III) sulfate, or other inorganic or organic chromium compounds. Preferably, the trivalent chromium compound is chromium (III) chloride hexahydrate, chromium (III) chloride, chromium (III) acetate, chromium (III) nitrate, chromium (III) oxide, or chromium (III) sulfate. More preferably, the trivalent chromium compound is chromium (III) chloride hexahydrate.

Optionally, the composition of the present disclosure may further comprise an active agent, an auxiliary agent, a dispersing agent, a wetting agent, an excipient, or a suspension agent.

In addition, the composition of the present disclosure may be prepared in any form, for example, powder, liquid, ready-to-eat sticks, capsules, liquid capsules, granules, tablets or dietary gel, and may be further processed in a conventional way, such as the addition of appropriate excipient.

The composition of the present disclosure may be prepared into the powder by the following method. For example, the composition of the present disclosure may be formed by mixing the powder of taurine, powder of trivalent chromium compound and powder of lactoferrin. Alternatively, pure water is added after mixing powder of taurine, powder of trivalent chromium compound and powder of lactoferrin, then a mixed solution is obtained after stirring, and the mixed solution is spray-dried to obtain the composition of the present disclosure. Alternatively, pure water is added after mixing powder of taurine, powder of trivalent chromium compound and powder of lactoferrin, then a mixed solution is obtained after stirring, the mixed solution is heated to 37° C. to 95° C. (preferably 40° C. to 60° C.), and the mixed solution is spray-dried to obtain the composition of the present disclosure. However, the present disclosure is not limited thereto.

The composition of the present disclosure may be prepared into the granule by the following method. For example, pure water is added after mixing the powder of taurine, powder of trivalent chromium compound and powder of lactoferrin, and then a mixed solution is obtained after stirring. Then, wet granulation is performed on the mixed solution by a moist granulator, wherein the temperature of the heater in the moist granulator is maintained between 70° C. to 80° C. Finally, the composition of the present disclosure is obtained. However, the present disclosure is not limited thereto.

The composition of the present disclosure may be prepared into a liquid form by the following method. For example, the powder of taurine is dissolved in the pure water, moderately heated to 40° C. to 60° C., and then the mixture is cooled down to 30° C. to 60° C. after the powder is completely dissolved. Then, the powder of lactoferrin and the powder of trivalent chromium compound are added into the mixture, and the composition of the present disclosure is obtained after stirring the mixture. When the composition of the present is prepared into the liquid form, it may be added into a liquid food having a pH value from 5.5 to 7.5 to be taken together with the liquid food. However, the present disclosure is not limited thereto.

In addition, the present disclosure further provides a method for promoting fat metabolism, which is particularly suitable for promoting fat metabolism in a subject in need. The method comprises the following steps: providing any aforementioned composition of the present disclosure to a subject in need thereof.

The present disclosure further provides a method for assisting in body weight control, which is particularly suitable for assisting in body weight control in a subject in need. The method comprises the following steps: providing any aforementioned composition of the present disclosure to a subject in need thereof.

The present disclosure further provides a method for promoting insulin activity, which is particularly suitable for promoting insulin activity in a subject in need. The method comprises the following steps: providing any aforementioned composition of the present disclosure to a subject in need thereof.

In any one of the aforementioned methods provided by the present disclosure, the “subject in need” may be a human or an animal, and the animal is preferably a mammal.

Furthermore, in the present disclosure, the aforementioned composition may be used as a dietary supplement to achieve the purpose of promoting insulin activity, promoting fat metabolism or assisting in body weight control. The dietary supplement may be added into and taken together with the liquid or solid dietary. For example, the dietary supplement may be mixed and consumed with water.

Therefore, the present application further provides a use of any one of the aforementioned composition of the present disclosure in the manufacture of a dietary supplement for promoting fat metabolism.

The present disclosure further provides a use of any one of the aforementioned compositions in the manufacture of a dietary supplement for assisting in body weight control.

The present disclosure further provides a use of any one of the aforementioned compositions in the manufacture of a dietary supplement for promoting insulin activity.

In addition, the present disclosure is not restrictive of the timing to take the composition of the aforementioned composition, and the timing can be changed to meet the needs of the subject in need. For instance, in the present disclosure, the aforementioned composition may be taken before a meal (for example, before breakfast or before dinner), and it may be taken before the meal by 30 to 60 minutes in advance, for example. However, the present disclosure is not limited thereto.

Other objects, advantages, and novel features of the invention will become more apparent from the following detailed description when taken in conjunction with the accompanying drawings.

### BRIEF DESCRIPTION OF THE DRAWINGS

FIG. 1 is a graph showing measurement results of the chromium element concentration accumulated in the tissues and organs of mice according to Test Example 1.

FIG. 2 is a graph showing the measurement result of the taurine concentration in the plasma of mice according to Test Example 1.

FIG. 3 is a graph showing changes in body weight of mice according to Test Example 2.

FIG. 4 is a graph showing the statistical results of the food intake and water intake of mice according to Test Example 2.

FIG. 5 is a graph showing the measurement results of the weight of mouse fat in Test Example 3.

FIG. 6 is a graph showing the measurement results of the triglyceride concentration in the serum of mice according to Test Example 4.

FIG. 7 is a graph showing the measurement results of the total cholesterol concentration in mice serum according to Test Example 4.

FIG. 8 is a graph showing the Western blot results of ACC and FAS in the livers of mice according to Test Example 5.

FIG. 9 is a graph showing quantitative results of ACC protein level in the mice liver according Test Example 5.

FIG. 10 is a graph showing quantitative results of FAS protein level in the mice liver according to Test Example 5.

### DETAILED DESCRIPTION OF THE PREFERRED EMBODIMENT

Unless otherwise specified, the singular terms “a” and “the” in the specification and the claims include one or plural individuals.

Unless otherwise specified, the term “or” in the specification and claims usually includes the meaning of “and/or”.

These embodiments are meant to explain the technical content of the present invention, but not meant to limit the scope of the present invention. Unless otherwise specified, the content used in the following examples to indicate the content of a material and the quantity of a substance is based on weight.

In the following test examples of the present disclosure, the mice in the control group are fed with low-fat milk powder containing no trivalent chromium complex and taurine; the mice in the trivalent chromium group are fed with low-fat milk powder containing the trivalent chromium complex; the mice in the trivalent chromium+taurine group are fed with low-fat milk powder containing trivalent chromium complex and taurine; and the mice in the taurine group are fed with low-fat milk powder containing taurine.

A preparation of the used composition containing the trivalent chromium complex is described as follows.

Preparation 1

Add lactoferrin (5 g) and chromium (III) chloride hexahydrate (0.1 g) to 1 L of pure water, and heat the mixture to 70° C. while stirring for two hours. After the cooling down procedure is finished, add 10 kg of milk powder and stir the mixture evenly, and then spray-dry the mixture to obtain the milk powder containing trivalent chromium complex.

A preparation of the used composition containing the trivalent chromium complex and taurine is described as follows.

Preparation 2

Add lactoferrin (5 g), taurine (3 kg) and chromium (III) chloride hexahydrate (0.1 g) into 3 L of pure water, heat the mixture to 70° C. while stirring for one hour, and stir the mixture for one hour after cooling down to 50° C. After the cooling down procedure is finished, add 7 kg of milk powder and stir the mixture evenly, and then spray-dry the mixture to obtain the milk powder containing trivalent chromium complex and taurine.

A preparation of the used composition containing taurine is described as follows.

Preparation 3

Add lactoferrin (5 g) and taurine (3 kg) into 3 L of pure water, heat the mixture to 70° C. while stirring for one hour, and stir the mixture for one hour after cooling down to 50° C. After the cooling down procedure is finished, add 7 kg of milk powder and stir the mixture evenly, and then spray-dry the mixture to obtain the milk powder containing taurine.

A preparation of the used composition containing no trivalent chromium and taurine is described as follows.

Preparation 4

Add lactoferrin (5 g) into 1 L of pure water, and heat the mixture to 70° C. while stirring for 2 hours. After the cooling down procedure is finished, add 10 kg of milk powder and stir the mixture evenly, and then spray-dry the mixture to obtain the milk powder containing no trivalent chromium and taurine.

### Test Example 1

Male C57BL/6JNarl mice were fed with high-fat diet (60% of calories derived from fat, D12492, Research Diets, New Brunswick, N.J., USA) to induce obesity, in which the substance to be tested was mixed with the mice diet. There were four groups with six mice in each group. The mice in the control group were fed with the placebo (i.e. the milk powder containing no trivalent chromium and taurine, prepared by Preparation 4). There were three experimental groups, which were trivalent chromium group, trivalent chromium+taurine group, and taurine group. The mice in the trivalent chromium group were fed with milk powder containing trivalent chromium complex, wherein the milk powder was prepared by Preparation 1 and contained 40 μg/kg BW/day of chromium. The mice in the trivalent chromium+taurine group were fed with milk powder containing trivalent chromium complex and taurine, wherein the milk powder was prepared by Preparation 2 and contained 40 μg/kg BW/day of chromium and 1.2 g/kg BW/day of taurine. The mice in the taurine group were fed with milk powder containing taurine, wherein the milk powder was prepared by Preparation 3 and contained 1.2 g/kg BW/day of taurine. The C57BL/6JNarl mice aged 8 weeks were fed for 8 weeks and sacrificed after eight weeks of continuous feeding. The mice tissues and organs were collected and analyzed for trace elements using an atomic absorption spectrometer. At the same time, one-way ANOVA was used for statistics, LSD method was used for comparison, in which P<0.05 indicated significant difference.

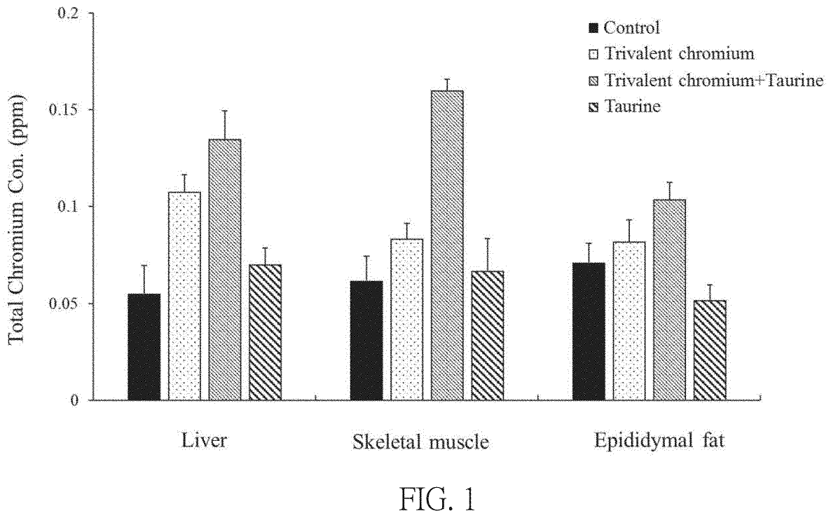

The results are shown in FIG. 1. In the trivalent chromium+taurine group, the concentration of chromium in the liver, muscle, and adipose tissue of the mice was significantly higher than that of other groups (p<0.05), indicating that the combination of trivalent chromium complex and taurine had synergistic effect on the chromium absorption efficiency and chromium accumulation efficiency. Because the liver, muscle, and fat are insulin-sensitive tissues, and play important roles in the glucose and lipid metabolism. Therefore, the concentration of chromium element accumulated in the insulin-sensitive tissue increases with time if the mice diet contains chromium element. In addition, the accumulation level relates to the absorption rate and excretion rate. Therefore, the higher concentration of chromium accumulating in insulin-sensitive tissues (for example, liver, muscle, and fat) indicates the better efficiency in accumulating chromium element. Accordingly, it is in favor of energy metabolism in body.

Meanwhile, the concentration of taurine in the plasma of the tested mice was also detected; and One-way ANOVA was used for statistics, and the LSD method was used for comparison, in which p<0.05 indicated a significant difference.

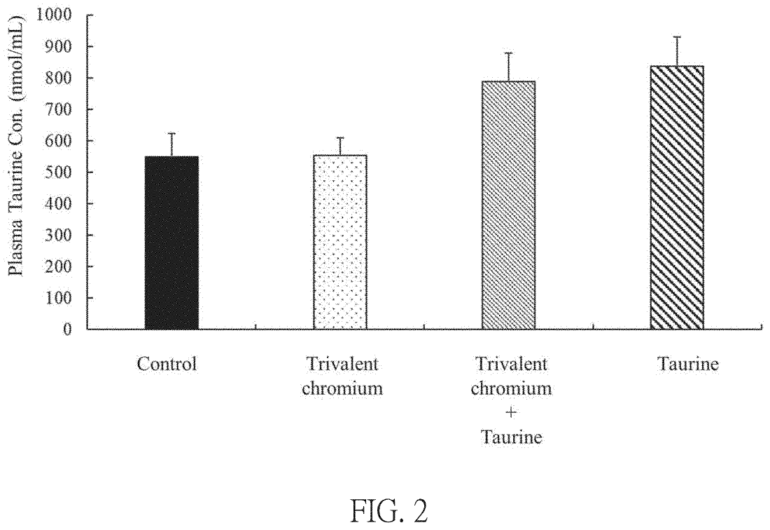

The results are shown in FIG. 2. In the group containing taurine, that is, the trivalent chromium+taurine group and the taurine group, the concentration of taurine in the plasma of mice is higher than the group containing no taurine (p<0.05). This result indicates that the taurine contained in the diet is involved in the metabolism of mice.

### Test Example 2

Male C57BL/6JNarl mice were fed with high-fat diet (60% of calories derived from fat, D12492, Research Diets, New Brunswick, N.J., USA) to induce obesity, in which the substance to be tested was mixed with the mice diet. There were four groups with six mice in each group. The mice in the control group were fed with the placebo (i.e. the milk powder containing no trivalent chromium and taurine, prepared by Preparation 4). There were three experimental groups, which were trivalent chromium group, trivalent chromium+taurine group, and taurine group. The mice in the trivalent chromium group were fed with milk powder containing trivalent chromium complex, wherein the milk powder was prepared by Preparation 1 and contained 40 μg/kg BW/day of chromium. The mice in the trivalent chromium+taurine group were fed with milk powder containing trivalent chromium complex and taurine, wherein the milk powder was prepared by Preparation 2 and contained 40 μg/kg BW/day of chromium and 1.2 g/kg BW/day of taurine. The mice in the taurine group were fed with milk powder containing taurine, wherein the milk powder was prepared by Preparation 3 and contained 1.2 g/kg BW/day of taurine. The C57BL/6JNarl mice aged 8 weeks were fed for 8 weeks and sacrificed after eight weeks of continuous feeding. The body weights of the tested mice were recorded every week. At the same time, one-way ANOVA was used for statistics, LSD method was used for comparison, in which P<0.05 indicated significant difference.

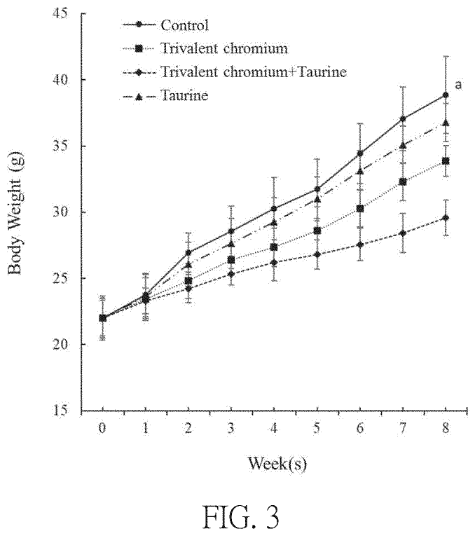

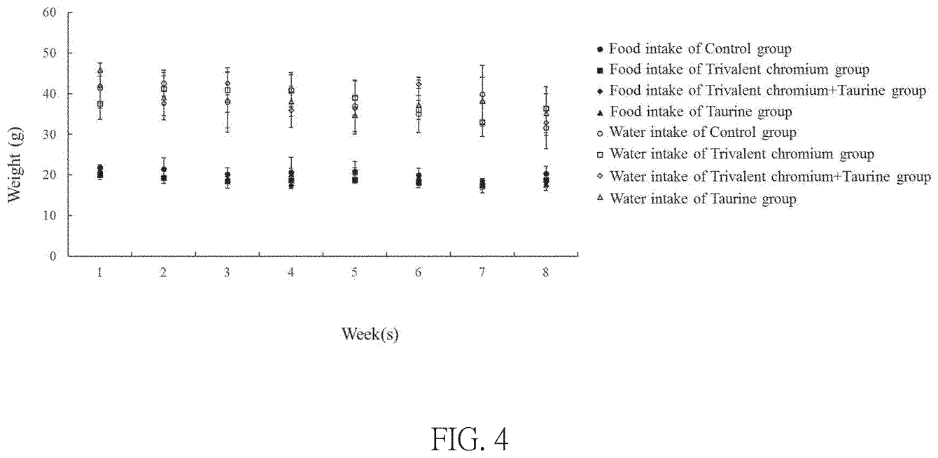

The results are shown in FIG. 3. The body weights of the mice in the trivalent chromium+taurine group were significantly less than those in the other groups during the period from the sixth week to the eighth week. In addition, the body weights of the mice in the trivalent chromium+taurine group increased by 25.6%, compared with the initial body weights. However, compared with the initial body weights, the body weights of the mice in the chromium group increased by 35.1%, the body weights of the mice in the taurine group increased by 40.2%, and the body weights of the mice in the control group increased by 43.4%, which were higher than the that of trivalent chromium+taurine group. These results prove that even a mouse is fed with high-fat diet, the body weight of the mouse can be controlled by using the trivalent chromium complex in combination with taurine. At the same time, the accumulation of chromium element in the insulin-sensitive tissue leads to the control of the body weight of a mouse. In addition, the food intake amount of mice in each group was measured weekly to confirm the change of the food intake amount did not contribute to the increase of body weight. As shown in FIG. 4, there was no statistically significant difference between the groups (p>0.05) in respect of weekly food intake amount. Similarly, drinking water has affect on the food intake amount for mice, however, there was no statistically significant difference between the groups (p>0.05) in respect of weekly water intake amount. Therefore, it can be found from the experimental results of food intake amount and water intake amount that the intakes of the mice did not have impact on the change in the body weights of the mice. These results further confirmed that the control for body weight of the mice in the trivalent chromium+taurine group was caused by the combined use of trivalent chromium complex and taurine.

### Test Example 3

Male C57BL/6JNarl mice were fed with high-fat diet (60% of calories derived from fat, D12492, Research Diets, New Brunswick, N.J., USA) to induce obesity, in which the substance to be tested was mixed with the mice diet. There were four groups with six mice in each group. The mice in the control group were fed with the placebo (i.e. the milk powder containing no trivalent chromium and taurine, prepared by Preparation 4). There were three experimental groups, which were trivalent chromium group, trivalent chromium+taurine group, and taurine group. The mice in the trivalent chromium group were fed with milk powder containing trivalent chromium complex, wherein the milk powder was prepared by Preparation 1 and contained 40 μg/kg BW/day of chromium. The mice in the trivalent chromium+taurine group were fed with milk powder containing trivalent chromium complex and taurine, wherein the milk powder was prepared by Preparation 2 and contained 40 μg/kg BW/day of chromium and 1.2 g/kg BW/day of taurine. The mice in the taurine group were fed with milk powder containing taurine, wherein the milk powder was prepared by Preparation 3 and contained 1.2 g/kg BW/day of taurine. The C57BL/6JNarl mice aged 8 weeks were fed for 8 weeks and sacrificed after eight weeks of continuous feeding. The epididymal fat was weighed to evaluate the body fat changes. At the same time, one-way ANOVA was used for statistics, LSD method was used for comparison, in which P<0.05 indicated significant difference.

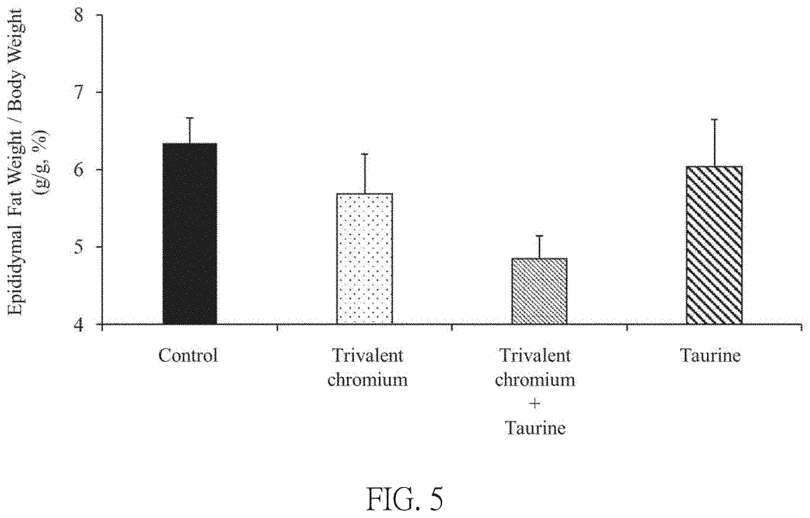

The results are shown in FIG. 5. The accumulation of chromium element in the insulin-sensitive tissues contributed to the control of fat metabolism, for example, the decrease in the weight of epididymal fat. As shown in FIG. 5, the weight of epididymal fat of the trivalent chromium+taurine group significantly decreased (p<0.05), indicating that the combined use of trivalent chromium complex and taurine had effect on reducing the formation of body fat. After calculation, the trivalent chromium+taurine group had less weight of epididymal fat than that of trivalent chromium group, taurine group and control group by 15.8%, 20.0% and 23.8%, respectively.

### Test Example 4

Male C57BL/6JNarl mice were fed with high-fat diet (60% of calories derived from fat, D12492, Research Diets, New Brunswick, N.J., USA) to induce obesity, in which the substance to be tested was mixed with the mice diet. There were four groups with six mice in each group. The mice in the control group were fed with the placebo (i.e. the milk powder containing no trivalent chromium and taurine, prepared by Preparation 4). There were three experimental groups, which were trivalent chromium group, trivalent chromium+taurine group, and taurine group. The mice in the trivalent chromium group were fed with milk powder containing trivalent chromium complex, wherein the milk powder was prepared by Preparation 1 and contained 40 μg/kg BW/day of chromium. The mice in the trivalent chromium+taurine group were fed with milk powder containing trivalent chromium complex and taurine, wherein the milk powder was prepared by Preparation 2 and contained 40 μg/kg BW/day of chromium and 1.2 g/kg BW/day of taurine. The mice in the taurine group were fed with milk powder containing taurine, wherein the milk powder was prepared by Preparation 3 and contained 1.2 g/kg BW/day of taurine. The C57BL/6JNarl mice aged 8 weeks were fed for 8 weeks and sacrificed after eight weeks of continuous feeding. Then, the concentrations of triglyceride and total cholesterol in the mouse serum were analyzed. At the same time, one-way ANOVA was used for statistics, and LSD method was used for comparison, in which P<0.05 indicated significant difference.

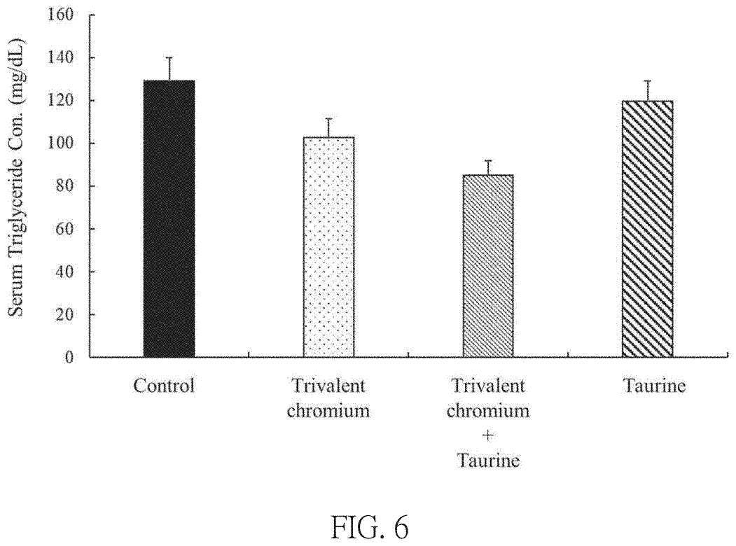

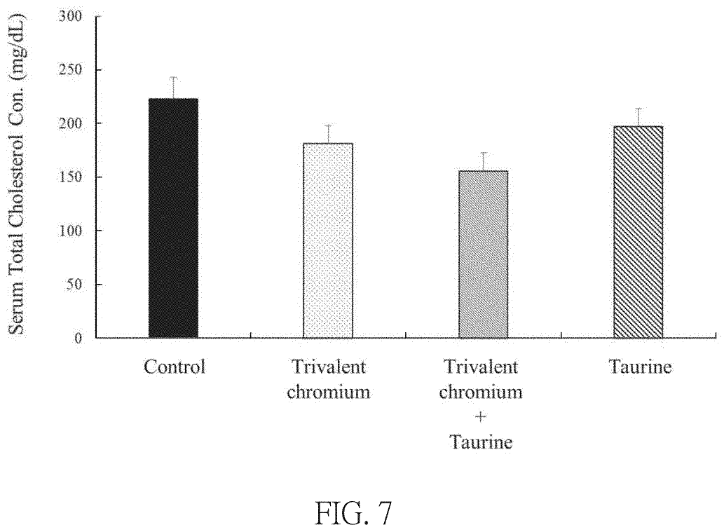

The measurement results of the triglyceride concentration and the total cholesterol concentration in the serum of the mice are shown in FIG. 6 and FIG. 7, respectively. As shown in FIG. 6, the triglyceride concentration in the serum of trivalent chromium+taurine group was significantly less than that of control group by 34.2% (p<0.05); and, the triglyceride concentrations in the serum of trivalent chromium group and taurine group were less than that of control group by 20.5% and 7.4%, respectively. As shown in FIG. 7, the total cholesterol concentration in the serum of trivalent chromium+taurine group was significantly less than that of control group by 30.1% (p<0.05); and, the total cholesterol concentrations in the serum of chromium group and taurine group were significantly less than that of control group by 18.5% and 11.5%, respectively.

### Test Example 5

Male C57BL/6JNarl mice were fed with high-fat diet (60% of calories derived from fat, D12492, Research Diets, New Brunswick, N.J., USA) to induce obesity, in which the substance to be tested was mixed with the mice diet. There were four groups with six mice in each group. The mice in the control group were fed with the placebo (i.e. the milk powder containing no trivalent chromium and taurine, prepared by Preparation 4). There were three experimental groups, which were trivalent chromium group, trivalent chromium+taurine group, and taurine group. The mice in the trivalent chromium group were fed with milk powder containing trivalent chromium complex, wherein the milk powder was prepared by Preparation 1 and contained 40 μg/kg BW/day of chromium. The mice in the trivalent chromium+taurine group were fed with milk powder containing trivalent chromium complex and taurine, wherein the milk powder was prepared by Preparation 2 and contained 40 μg/kg BW/day of chromium and 1.2 g/kg BW/day of taurine. The mice in the taurine group were fed with milk powder containing taurine, wherein the milk powder was prepared by Preparation 3 and contained 1.2 g/kg BW/day of taurine. The C57BL/6JNarl mice aged 8 weeks were fed for 8 weeks and sacrificed after eight weeks of continuous feeding. The tissues and organs of the mice were collected and subjected to western blot analysis.

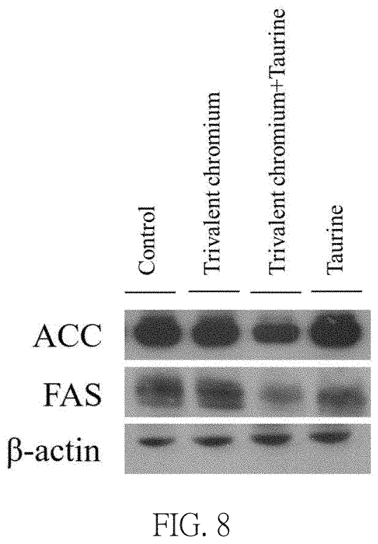

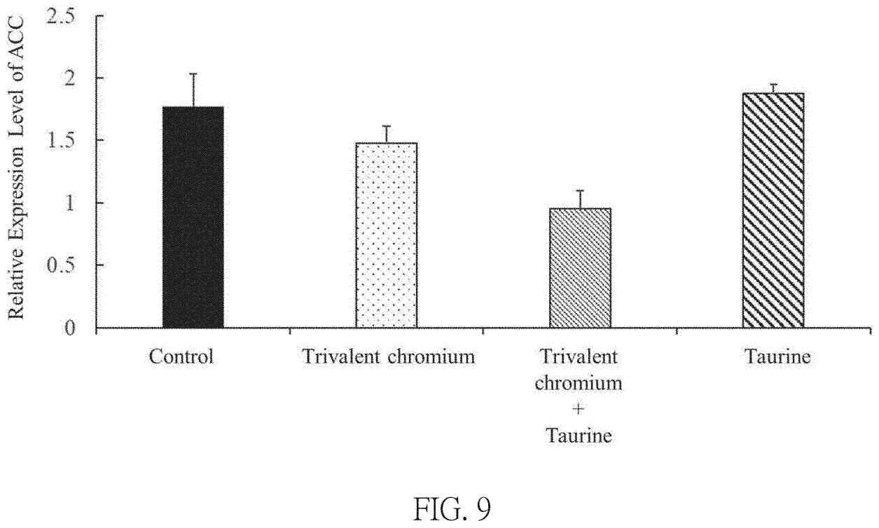

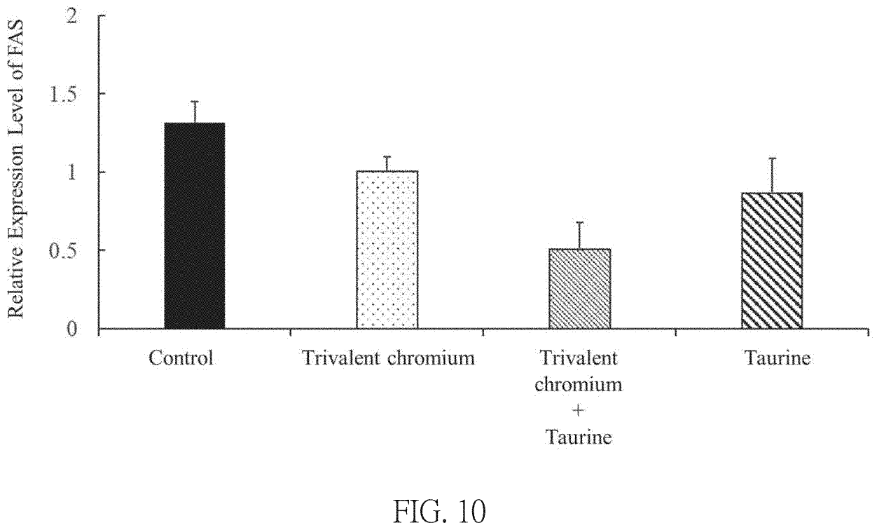

The results of western blot is shown in FIG. 8, and the quantitative results of acetyl-CoA carboxylase (ACC) and fatty acid synthase (FAS) are shown in FIG. 9 and FIG. 10. As shown in FIGS. 8 to 10, the protein activity of ACC or FAS in the liver can be effectively controlled by using the trivalent chromium complex in combination with taurine, thereby affecting lipid metabolism to reduce the formation of lipid. In particular, the trivalent chromium+taurine group can effectively inhibit the protein activity of ACC or FAS in the liver (p<0.05), as shown in FIG. 9 and FIG. 10.

According to the results of Test Examples 1 to 5, the composition containing trivalent chromium complex and taurine of the present disclosure can promote the accumulation of chromium element in the insulin-sensitive tissues, effectively control the protein activity of ACC or FAC, and effectively reduce the concentration of triglyceride or total cholesterol. The accumulation of chromium element in the insulin-sensitive tissues can further avoid or inhibit the formation of lipid in order to achieve the purpose of body weight control.

Although the present disclosure has been explained in relation to its preferred embodiment, it is to be understood that many other possible modifications and variations can be made without departing from the spirit and scope of the invention as hereinafter claimed.

What is claimed is:

1. A method for promoting lipid metabolism, comprising:
providing a composition to a subject in need thereof, wherein the composition comprises: a trivalent chromium complex being a complex of a trivalent chromium compound and a lactoferrin; and
a taurine or a derivative thereof.

2. The method according to claim 1, wherein a content of chromium in the trivalent chromium complex is in a range from 0.1 to 1 parts by weight, and a content of the taurine or the derivative thereof is in a range from 1,000 to 10,000 parts by weight.

3. The method according to claim 1, wherein a content of chromium in the trivalent chromium compound is in a range from 0.1 to 1 parts by weight, a content of lactoferrin is in a range from 1 to 20 parts by weight, and a content of the taurine or the derivate thereof is in a range from 1,000 to 10,000 parts by weight.

4. The method according to claim 1, wherein the composition further comprises: 1,000 to 50,000 parts by weight of glutamine.

5. The method according to claim 1, wherein the composition further comprises: 100 to 5,000 parts by weight of cysteine.

6. The method according to claim 1, wherein the composition further comprises: 300 to 5,000 parts by weight of glycine.

7. The method according to claim 1, wherein the composition further comprises: 1 to 30 parts by weight of vitamin E, 0.1 to 10 parts by weight of vitamin B6, or 1,000 to 30,000 parts by weight of whey protein.

8. The method according to claim 1, wherein the trivalent chromium compound is chromium (III) chloride hexahydrate, chromium (III) chloride, chromium (III) acetate, chromium (III) nitrate, chromium (III) oxide, or chromium (III) sulfate.

9. The method according to claim 8, wherein the trivalent chromium compound is chromium (III) chloride hexahydrate.

10. A method for assisting in body weight control, comprising:
providing a composition to a subject in need thereof, wherein the composition comprises: a trivalent chromium complex being a complex of a trivalent chromium compound and a lactoferrin; and
a taurine or a derivative thereof.

11. The method according to claim 10, wherein a content of chromium in the trivalent chromium complex is in a range from 0.1 to 1 parts by weight, and a content of the taurine or the derivative thereof is in a range from 1,000 to 10,000 parts by weight.

12. The method according to claim 10, wherein a content of chromium in the trivalent chromium compound is in a range from 0.1 to 1 parts by weight, a content of lactoferrin is in a range from 1 to 20 parts by weight, and a content of the taurine or the derivate thereof is in a range from 1,000 to 10,000 parts by weight.

13. The method according to claim 10, wherein the composition further comprises: 1,000 to 50,000 parts by weight of glutamine.

14. The method according to claim 10, wherein the composition further comprises: 100 to 5,000 parts by weight of cysteine.

15. The method according to claim 10, wherein the composition further comprises: 300 to 5,000 parts by weight of glycine.

16. The method according to claim 10, wherein the composition further comprises: 1 to 30 parts by weight of vitamin E, 0.1 to 10 parts by weight of vitamin B6, or 1,000 to 30,000 parts by weight of whey protein.

17. The method according to claim 10, wherein the trivalent chromium compound is chromium (III) chloride hexahydrate, chromium (III) chloride, chromium (III) acetate, chromium (III) nitrate, chromium (III) oxide, or chromium (III) sulfate.

18. The method according to claim 17, wherein the trivalent chromium compound is chromium (III) chloride hexahydrate.
